(19) United States
(12) Patent Application Publication
(10) Pub. No.: US 2023/0000589 A1
(43) Pub. Date: Jan. 5, 2023

(54) APPARATUS AND METHOD FOR DISCRIMINATING PROBES USING RESONANT FREQUENCY ACCORDING TO ACOUSTIC IMPEDANCE

(71) Applicant: NEWPONG Co., Ltd, Wonju-si (KR)

(72) Inventor: Jaeyong LEE, Seoul (KR)

(21) Appl. No.: 17/831,107

(22) Filed: Jun. 2, 2022

(30) Foreign Application Priority Data
Jul. 1, 2021 (KR) 10-2021-0086434

(51) Int. Cl. A61B 90/90; A61B 17/32

(52) U.S. Cl. CPC A61B 90/90; A61B 17/320068; A61B 2017/00084

(57) ABSTRACT

Provided is a method of discriminating probes by using a change in resonant frequency, the method including receiving an identification frequency for discriminating the type of probe of an ultrasonic surgical device, analyzing a feedback signal generated on the basis of the received identification frequency information, discriminating any one probe among multiple types of probes on the basis of comparison between an analysis result and preset reference frequency information, and generating a control signal for controlling an operation of the ultrasonic surgical device on the basis of a predetermined value corresponding to the discriminated type of probe.

### BACKGROUND

### Field

The present application relates to an apparatus and method for discriminating probes using a resonant frequency according to acoustic impedance.

### Description of the Related Art

In general, ultrasonic waves refer to acoustic waves having frequencies higher than 20 kHz exceeding ranges of audible frequencies audible to the human ears. The ultrasonic waves are widely used for ultrasonic imaging devices that obtain images of interiors of objects. The advantages of the ultrasonic imaging device are that the ultrasonic imaging device is small in scale and low in cost, the ultrasonic imaging device displays information in real time, and the ultrasonic imaging device has high stability because there is no exposure to radiation such as X-rays. Therefore, the ultrasonic imaging devices are widely used together with other imaging diagnosis devices such as computed tomography (CT) devices, magnetic resonance imaging (MRI) devices, and nuclear medicine device.

Recently, technologies related to ultrasonic therapeutic systems, together with the ultrasonic imaging devices, are also being actively developed. Most ultrasonic therapeutic systems obtain therapeutic effects by generating vibration of tissue or heat in a body by irradiating the body with the ultrasonic waves having frequencies of several MHz and used for medical purposes. A representative example of the ultrasonic therapeutic system is a high-intensity focused ultrasound system. In general, the high-intensity focused ultrasound system is embedded with a transducer configured to emit the ultrasonic waves and focuses the emitted ultrasonic waves on a focal point to generate heat, thereby rapidly raising a temperature of a surgical site. An intended surgical procedure is performed on various types of diseased parts by the heating function without leaving adverse effect.

The ultrasonic surgical device uses various types of probes respectively suitable for surgical sites and patient's characteristics, and the settings of the ultrasonic surgical device need to be adjusted depending on the type of probe. For this reason, there is an inconvenience in that a user needs to discriminate the probes being used and input information of the probes to the apparatus.

The background art of the present application is disclosed in Korean Patent No. 10-1605527.

### SUMMARY

The present application has been made in an effort to solve the above-mentioned problems in the related art, and an object of the present disclosure is to provide an apparatus for discriminating probes using a resonant frequency according to acoustic impedance, which is capable of automatically discriminating types of probes and selecting settings of the probes.

However, technical problems to be solved by the exemplary embodiment of the present application are not limited to the aforementioned technical problem, and other technical problems may be present.

According to an aspect of the present disclosure, there is provided a method for discriminating probes using a resonant frequency according to acoustic impedance, the method including: receiving an identification frequency for discriminating the type of probe of an ultrasonic surgical device; analyzing a feedback signal generated on the basis of the received identification frequency information; discriminating any one probe among multiple types of probes on the basis of comparison between an analysis result and preset reference frequency information; and generating a control signal for controlling an operation of the ultrasonic surgical device on the basis of a predetermined value corresponding to the discriminated type of probe.

In addition, the discriminating of the probe may include: detecting information on a temperature generated by an ultrasonic vibrator included in the ultrasonic surgical device; and correcting frequency error information according to a change in temperature by adjusting frequency information on the basis of a difference between pre-stored temperature change frequency information and the identification frequency information, and any one probe may be determined among the multiple types of probes in consideration of the corrected frequency information.

In addition, the method may include: before the receiving of the identification frequency, a tuning step of scanning a frequency to find a resonant frequency of a transducer included in the ultrasonic surgical device; and a tracking step of maintaining the resonant frequency of the transducer, which is detected by scanning, as a predetermined resonant frequency, and the receiving of the identification frequency may include receiving identification frequency detected in the tuning step and the tracking step.

According to another aspect of the present disclosure, there is provided an apparatus for discriminating probes using a change in resonant frequency, the apparatus including: a receiving unit configured to receive an identification frequency for discriminating the type of probe of an ultrasonic surgical device including a transducer and configured to generate ultrasonic waves by using an ultrasonic vibrator on the basis of a control signal; an analyzing unit configured to analyze a feedback signal generated on the basis of the received identification frequency information; a discriminating unit configured to discriminate any one probe among multiple types of probes on the basis of comparison between an analysis result and preset reference frequency information; and a drive control unit configured to generate a control signal for controlling an operation of the ultrasonic surgical device on the basis of a predetermined value corresponding to the discriminated type of probe.

In addition, the discriminating unit may include: a temperature detecting unit configured to detect information on a temperature generated by the ultrasonic vibrator included in the ultrasonic surgical device; and a correcting unit configured to correct frequency error information according to a change in temperature by adjusting frequency information on the basis of a difference between pre-stored temperature change frequency information and the identification frequency information, and any one probe may be determined among the multiple types of probes in consideration of the corrected frequency information.

The technical solution is just illustrative but should not be interpreted as being intended to limit the present application. In addition to the above-mentioned exemplary embodiment, additional exemplary embodiments may be present in the drawings and the detailed description of the invention.

According to the technical solutions according to the present application, it is possible to automatically discriminate the types of probes of the ultrasonic surgical device and select the setting suitable for the probe, thereby providing the convenience.

According to the technical solutions according to the present application, the probe may be designed such that the probe efficiently vibrates at a desired frequency so that the probes may be discriminated.

However, the effects, which can be obtained by the present application, are not limited to the above-mentioned effects, and other effects may be present.

### BRIEF DESCRIPTION OF THE DRAWINGS

The above and other aspects, features and other advantages of the present disclosure will be more clearly understood from the following detailed description taken in conjunction with the accompanying drawings, in which:

FIG. 1 is an operational flowchart illustrating a method of discriminating types of probes of an ultrasonic surgical device according to an embodiment of the present application;

FIG. 2 is a view schematically illustrating an oscillation frequency for each of the probes of the ultrasonic surgical device according to the embodiment of the present application; and

FIG. 3 is a schematic block diagram of an apparatus for discriminating probes according to the embodiment of the present application.

### DETAILED DESCRIPTION OF THE EMBODIMENT

Hereinafter, exemplary embodiments of the present application will be described in detail with reference to the accompanying drawings so that those with ordinary skill in the art to which the present application pertains may easily carry out the exemplary embodiments. However, the present application may be implemented in various different ways, and is not limited to the exemplary embodiments described herein. A part irrelevant to the description will be omitted in the drawings in order to clearly describe the present application, and similar constituent elements will be designated by similar reference numerals throughout the specification.

Throughout the specification of the present application, when one constituent element is referred to as being “connected to” another constituent element, one constituent element can be “directly connected to” the other constituent element, and one constituent element can also be “electrically connected to” or “indirectly connected to” the other element with other elements therebetween.

Throughout the specification, when one member is disposed “on”, “at an upper side of”, “at an upper end of”, “below”, “at a lower side of”, or “at a lower end of” another member in the present specification of the present application, this includes not only a case where one member is brought into contact with another member, but also a case where still another member is present between the two members.

Throughout the specification of the present application, unless explicitly described to the contrary, the word “comprise” or “include” and variations, such as “comprises”, “comprising”, “includes” or “including”, will be understood to imply the inclusion of stated constituent elements, not the exclusion of any other constituent elements.

The present application relates to an apparatus and method for discriminating probes using a change in resonant frequency according to acoustic impedance.

An ultrasonic surgical device uses various types of probes respectively suitable for surgical sites and patient's characteristics, and the settings of the ultrasonic surgical device need to be adjusted depending on the type of probe. A user may discriminate the probes being used by inputting the information on the probes to the apparatus. Further, the ultrasonic surgical device may be more efficiently used when the apparatus may automatically discriminate types of probes and select the settings suitable for the probes.

Meanwhile, the probe may be designed such that the probe efficiently vibrates at a desired frequency so that the probes may be discriminated. Diameters of the probes may be predetermined, and lengths and groove shapes may be modified and adopted to the design.

Hereinafter, the apparatus for discriminating probes using a change in resonant frequency according to acoustic impedance will be referred to as the present apparatus 100.

The present apparatus 100 may serve to automatically identify various types of probes and identify any one probe among the multiple types of probes by using the fact that the ultrasonic oscillation varies depending on a thickness and a length of the probe.

In addition, the present apparatus 100 may analyze a feedback signal related to an ultrasonic output and identify the type of probe by using the fact that frequencies at which the types of probes most efficiently vibrate are different from one another.

The ultrasonic waves of the ultrasonic surgical device to be described below may mean, but not limited to, the generation of ultrasonic vibration that serves as a surgical knife (scalpel) for a surgical procedure (e.g., incision).

The ultrasonic surgical device generates acoustic vibration having non-audible frequencies of 20 kHz or higher by using an ultrasonic vibration device (BLT). In this case, the ultrasonic surgical device uses generated mechanical kinetic energy and frictional heat to incise and coagulate tissue during various types of surgical procedures.

Most of medical ultrasonic surgical devices use an output of about 100 to 200 W and use an ultrasound handpiece having oscillating frequencies within a range of 20 to 60 kHz depending on the use of the surgical procedure. The ultrasonic surgical device uses a low-frequency probe when large amplitude (amplitude of about 300 μm) is required, and uses a high-frequency probe (with amplitude of about 60 μm to 80 μm) when the ultrasonic surgical device needs to perform a delicate surgical procedure.

The amplitude varies depending on driving power and an ultrasound horn shape suitable for the ultrasonic frequency. However, as the frequency increases, a size and weight of the ultrasound handpiece decrease and the ultrasound handpiece is miniaturized (kinetic energy of the ultrasonic waves increases as the amplitude and the frequency increase).

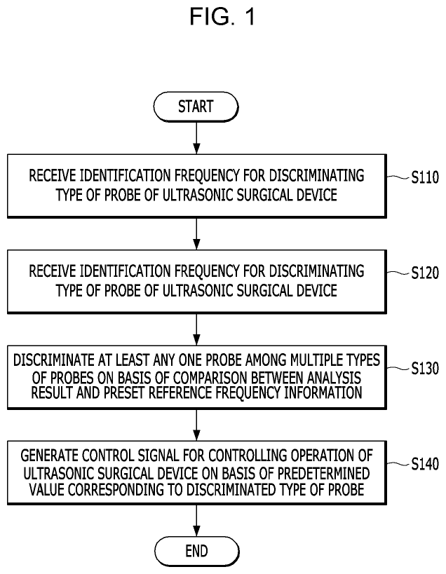

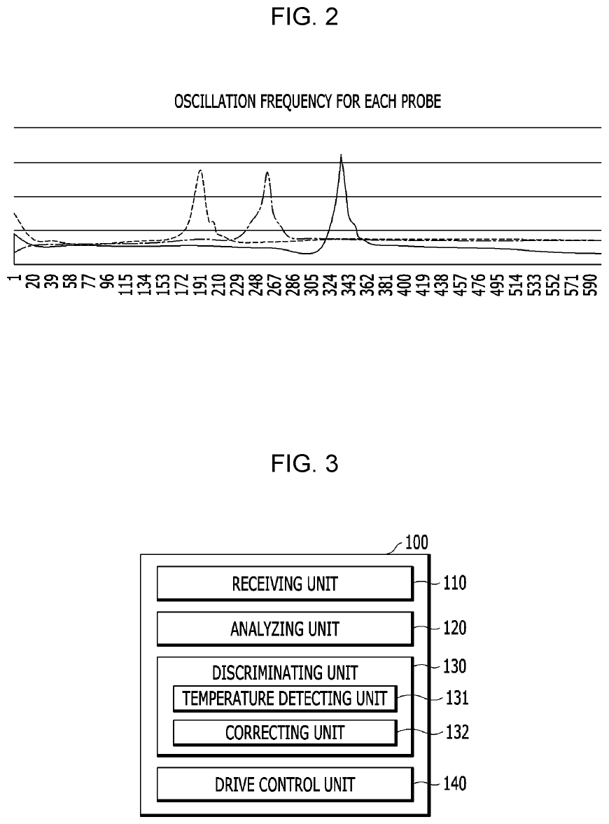

FIG. 1 is an operational flowchart illustrating a method of discriminating types of probes of an ultrasonic surgical device according to an embodiment of the present application, and FIG. 2 is a view schematically illustrating an oscillation frequency for each of the probes of the ultrasonic surgical device according to the embodiment of the present application.

Hereinafter, for the convenience of description, a resonant frequency control system 1 of the ultrasonic surgical device will be referred to as the present system 1.

The present system 1 may control the resonant frequency of the ultrasonic surgical device capable of performing tuning and tracking within a preset time on the basis of a signal for operating the ultrasonic surgical device inputted by a user without a separate tuning process.

In addition, the present system 1 may obtain an inherent resonant frequency of a transducer included in the ultrasonic surgical device and maintain an optimal resonant frequency through the tuning and tracking processes.

In addition, an ultrasonic surgical device in the related art performs the tuning process by manipulating a separate switch (e.g., a switch for performing the tuning process). In contrast, the present system 1 may immediately initiate the operation without requiring a separate manipulation for tuning.

The present system 1 may provide the present apparatus 100 with an identification frequency for discriminating the type of probe of the ultrasonic surgical device. The present apparatus 100 may discriminate any one probe among the multiple types of probes by analyzing a feedback signal produced on the basis of the identification frequency and generate a control signal suitable for the corresponding probe. The present system 1 may control the operation of the ultrasonic surgical device by receiving the corresponding control signal.

In step S110, the present apparatus 100 may receive the identification frequency for discriminating the type of probe of the ultrasonic surgical device. In other words, the present apparatus 100 may receive a resonant frequency detected (e.g., predicted) by the present system 1 as the identification frequency. The identification frequency may be used to discriminate the type of probe.

The present system 1 may scan frequencies in order to find the resonant frequency of the transducer included in the ultrasonic surgical device. In addition, the present system 1 may maintain the resonant frequency of the transducer, which is detected by scanning, as a predetermined resonant frequency.

In other words, the present system 1 may scan the identification frequency for finding the resonant frequency of the particular probe on the basis of an operating signal of an output mode provided from a foot switch. The present system 1 may perform the process of scanning the identification frequency in order to find the resonant frequency of the transducer included in the ultrasonic surgical device.

For example, the present system 1 may use a direct digital synthesizer (DDS) and change the frequency by increasing the frequency by a preset frequency value (e.g., 4 kHz) from a first frequency (e.g., 30 kHz) to a second frequency (e.g., 32 kHz). In this case, the present system 1 may determine a frequency, which induces maximum voltage, as the resonant frequency by monitoring a voltage inputted through a feedback circuit.

For example, an ultrasonic vibrator generates ultrasonic waves on the basis of the resonant frequency. The ultrasonic vibrator may match the changing resonant frequency with the oscillating frequency and maintain the constant output of the ultrasonic waves, thereby improving the function of the ultrasonic surgical device.

In addition, the present system 1 may perform the tracking process of maintaining the resonant frequency of the transducer, which is detected by scanning, as a predetermined (optimal) resonant frequency. The tracking process may monitor the voltage inputted through the feedback circuit for each preset time (e.g., 100 to 200 ms), compare the monitored voltage with the voltage detected during the tuning process, and adjust and correct the frequency depending on a difference in voltage.

For example, the present system 1 may decrease the frequency when the voltage decreases, and increase the frequency when the voltage increases. For example, the present system 1 may predict the resonant frequency within a particular range of the frequency of the transducer of the ultrasonic surgical device (ultrasonic probe).

In addition, the present system 1 may compare the frequency at the maximum voltage with the current frequency. For example, the present system 1 may detect the maximum voltage and detect the frequency at the maximum voltage. In addition, the present system 1 may detect the current frequency generated by the transducer of the ultrasonic therapeutic device (ultrasonic probe).

The present system 1 may determine whether the maximum voltage frequency is within a preset range. In this case, the maximum voltage frequency may be the resonant frequency. In this case, when the maximum voltage frequency is within the preset range, the present system 1 may perform the tracking process in response to feedback. In contrast, when the maximum voltage frequency is not within the preset range, the present system 1 may perform the tuning process again. In other words, when the result of the comparison between the frequency at the maximum voltage and the current frequency is not within the preset range, the present system 1 may generate a feedback signal related to a process of performing a tuning step again.

For example, the present system 1 may include a retuning step of scanning the frequency again in order to find the resonant frequency of the transducer included in the ultrasonic surgical device on the basis of the feedback signal after a tracking step. For example, the present system 1 may perform the retuning process of scanning the frequency again to find the resonant frequency of the transducer included in the ultrasonic therapeutic device on the basis of the feedback signal after the tracking step without performing the tuning process by the user's input and the manipulation of the separate switch.

In step S120, the present apparatus 100 may analyze the feedback signal generated on the basis of the received information on the identification frequency. The present apparatus 100 may analyze the feedback related to the output of the ultrasonic waves by using the fact that the ultrasonic oscillation varies depending on the thickness and length of the probe. The present apparatus 100 may analyze the feedback signal generated by the present system 1 to find the resonant frequency.

In step S130, the present apparatus 100 may discriminate any one probe among the multiple types of probes by comparing the analysis result with preset reference frequency information. For example, referring to FIG. 2, because the frequencies at which the types of probes most efficiently vibrate are different from one another, it is possible to discriminate the probes by analyzing the feedback signal and the reference frequency information according to the preset type of probe.

The present apparatus 100 may discriminate any one probe among the multiple types of probes by comparing the analysis result with the reference frequency information stored in database (not illustrated).

The database (not illustrated) may store first reference frequency information corresponding to a first probe, second reference frequency information corresponding to a second probe, and third reference frequency information corresponding to a third probe.

The first to third probes may be different in thicknesses, lengths, and groove shapes. The first reference frequency information and the third reference frequency information may be information corresponding to the frequency at which the particular probe most efficiently vibrates.

In addition, the present apparatus 100 may detect information on a temperature produced by the ultrasonic vibrator included in the ultrasonic surgical device. The maximum output is made at the resonant frequency, but a change in impedance may occur, for example, within a range of 10 to 20% because of a change in temperature during the operating process. For this reason, the resonance state may not be maintained because of an increase in temperature.

The present apparatus 100 is intended to solve the problem in that the discrimination deteriorates at the time of discriminating the probes because the characteristics of the ultrasonic vibrator are changed by the temperature of the ultrasonic vibrator. Therefore, the present apparatus 100 may discriminate the types of probes after measuring the temperature of the ultrasonic vibrator and correcting the frequency information on the basis of information related to the temperature of the ultrasonic vibrator.

In addition, the present apparatus 100 may correct frequency error information according to the change in temperature by adjusting the frequency information on the basis of the difference between the pre-stored temperature change frequency information and the identification frequency information.

For example, the present apparatus 100 may correct the frequency error information according to the change in temperature by adjusting the frequency information on the basis of the difference between the identification frequency information and the temperature change frequency information stored in the database.

The database may store characteristic information of the vibrator that changes at a first temperature, characteristic information of the vibrator that changes at a second temperature, and characteristic information of the vibrator that changes at a third temperature.

The present apparatus 100 may correct the error information by adjusting the frequency information on the basis of the difference between the identification frequency information and the frequency information that changes at a particular temperature stored in advance in the database. In other words, the present apparatus 100 may track a change in resonant frequency of the transducer caused by a change in impedance or heating as the time elapses during the operation and perform the correction so that the corresponding probe may maintain the optimal resonant frequency.

The present apparatus 100 may determine any one probe among the multiple types of probes on the basis of the optimal resonant frequency information generated by the corresponding probe with reference to the corrected frequency error information.

In step S140, the present apparatus 100 may generate a control signal for controlling the ultrasonic surgical device on the basis of a predetermined value corresponding to the discriminated type of probe. For example, the control signal generated by the present apparatus 100 may be a generated signal related to the frequencies, strength, and generation types of ultrasonic waves.

For example, when the discriminated type of probe is the first probe, the present apparatus 100 may generate a control signal for generating ultrasonic waves having a first frequency band. In addition, when the discriminated type of probe is the second probe, the present apparatus 100 may generate a control signal for generating ultrasonic waves having a second frequency band. In addition, when the discriminated type of probe is the third probe, the present apparatus 100 may generate a control signal for generating ultrasonic waves having a third frequency band.

For example, the second frequency band may be a frequency with a low band based on the first frequency. In addition, the third frequency band may be a frequency with a high band based on the first frequency.

As another example, when the discriminated type of probe is the first probe, the present apparatus 100 may generate a first control signal, and the first control signal may be a signal corresponding to the first frequency band, first ultrasonic strength, and a first generation type of ultrasonic waves. In addition, when the discriminated type of probe is the second probe, the present apparatus 100 may generate a second control signal, and the second control signal may be a signal corresponding to the second frequency band, second ultrasonic strength, and a second generation type of ultrasonic waves.

The present apparatus 100 may receive the identification frequency from the present system 1 and generate the control signal for controlling a predetermined value of the particular probe on the basis of information on the type of probe determined by analyzing the feedback signal related to the output of ultrasonic waves.

Hereinafter, the present system 1 capable of controlling the resonant frequency of the ultrasonic surgical device in consideration of the control signal provided from the present apparatus 100 will be described. When the present apparatus 100 determines the particular probe, the resonant frequency corresponding to (suitable for) the corresponding probe may be determined, and the present system 1 may generate the control signal for maintaining the resonant frequency corresponding to the corresponding probe.

The present system 1 may initiate the tuning step and the tracking step on the basis of the operating signal of the output mode provided from the foot switch. For example, the tuning process scans the frequency to find the resonant frequency of the transducer. The tracking process tracks, in real time, a change in resonant frequency of the transducer, which is detected by scanning, caused by a change in impedance or heating as the time elapses during the operation and maintains the optimal resonant frequency.

The present system 1 may initiate the tuning step and the tracking step on the basis of a signal for initiating the ultrasonic surgical device (ultrasonic probe) and a switch for starting ultrasonic therapy instead of an operation of a separate switch for performing the tuning and tracking steps.

According to the embodiment of the present application, the present system 1 may control the operation of the ultrasonic surgical device by using a control signal generated to control the operation of the ultrasonic surgical device on the basis of a predetermined value corresponding to the type of probe discriminated by the present apparatus 100.

The present system 1 may scan the resonant frequency of the transducer on the basis of an output level inputted from a rotary switch and the operating signal of the output mode inputted from the foot switch. For example, the present system 1 may set the output level corresponding to (suitable for) the corresponding probe on the basis of the control signal provided from the present apparatus 100. In addition, the present system 1 may select the output mode including a repetitive (continuous) mode and a rhythm (pulse) mode corresponding to the corresponding probe on the basis of the control signal provided from the present apparatus 100.

In addition, when the result of comparing the current frequency and the frequency at the maximum voltage is not within a preset range, the present system 1 may generate a feedback signal for performing the tuning step again.

In addition, when the result of comparing the current frequency and the frequency at the maximum voltage is within the preset range, the present system 1 may generate a feedback signal for performing the tracking step again. The present apparatus 100 may analyze the feedback signal generated by the present system 1.

FIG. 3 is a schematic block diagram of an apparatus for discriminating probes according to the embodiment of the present application.

Referring to FIG. 3, the apparatus 100 for discriminating probes (the present apparatus) may include a receiving unit 110, an analyzing unit 120, a discriminating unit 130, and a drive control unit 140.

According to the embodiment of the present application, the receiving unit 110 may receive the identification frequency for discriminating the type of probe of the ultrasonic surgical device. The receiving unit 110 may receive the identification frequency measured by the present system 1 by the operation of the ultrasonic therapeutic device.

The identification frequency may be the frequency related to the frequency scanning process performed to find the resonant frequency of the transducer included in the ultrasonic surgical device in the present system 1. In addition, the receiving unit 110 may receive information on the resonant frequency determined by the present system 1 as the identification frequency.

In addition, the analyzing unit 120 may analyze the feedback signal generated on the basis of the received identification frequency information. The analyzing unit 120 may analyze the feedback signal generated to generate the resonant frequency in the particular probe. The feedback signal may be a signal generated to generate the optimal resonant frequency in the corresponding probe.

In addition, the discriminating unit 130 may discriminate any one probe among the multiple types of probes on the basis of the comparison between the analysis result and the preset reference frequency information.

The discriminating unit 130 may include a temperature detecting unit 131 and a correcting unit 132. The temperature detecting unit 131 may detect information on the temperature generated in the ultrasonic vibrator 21 included in the ultrasonic surgical device.

The correcting unit 132 may correct frequency error information according to the change in temperature by adjusting the frequency information on the basis of the difference between the pre-stored temperature change frequency information and the identification frequency information.

In addition, the drive control unit 140 may generate a control signal for controlling the ultrasonic surgical device on the basis of a predetermined value corresponding to the discriminated type of probe. In this case, the control signal may be a drive control signal that may be generated to correspond to the particular probe.

The method of discriminating probes according to the embodiment of the present application may be implemented in the form of program commands executable by means of various computer means and then written in a computer-readable recording medium. The computer-readable medium may include program instructions, data files, data structures, or the like, in a stand-alone form or in a combination thereof. The program instructions recorded in the medium may be specially designed and configured for the present disclosure or may be known and available to those skilled in computer software. Examples of the computer-readable recording medium may include magnetic media, such as a hard disk, a floppy disk and a magnetic tape, optical media, such as CD-ROM and DVD, magneto-optical media, such as a floptical disk, and hardware devices, such as ROM, RAM and flash memory, which are specifically configured to store and run program instructions. Examples of the program instructions may include machine codes made by, for example, a compiler, as well as high-language codes that may be executed by an electronic data processing device, for example, a computer, by using an interpreter. The above-mentioned hardware devices may be configured to operate as one or more software modules in order to perform the operation of the present disclosure, and the opposite is also possible.

In addition, the method of discriminating probes may also be implemented in the form of a computer program or application stored in a recording medium and executed by a computer.

It will be appreciated that the exemplary embodiments of the present application have been described above for purposes of illustration, and those skilled in the art may understand that the present application may be easily modified in other specific forms without changing the technical spirit or the essential features of the present application. Therefore, it should be understood that the above-described embodiments are illustrative in all aspects and do not limit the present disclosure. For example, each component described as a single type may be carried out in a distributed manner. Likewise, components described as a distributed type can be carried out in a combined type.

The scope of the present application is represented by the claims to be described below rather than the detailed description, and it should be interpreted that the meaning and scope of the claims and all the changes or modified forms derived from the equivalent concepts thereto fall within the scope of the present application.

What is claimed is:

1. A method of discriminating probe, the method comprising:
receiving an identification frequency for discriminating the type of probe of an ultrasonic surgical device;
analyzing a feedback signal generated on the basis of the received identification frequency information;
discriminating any one probe among multiple types of probes on the basis of comparison between an analysis result and preset reference frequency information; and
generating a control signal for controlling an operation of the ultrasonic surgical device on the basis of a predetermined value corresponding to the discriminated type of probe.

2. The method of claim 1, wherein the discriminating of the probe comprises:
detecting information on a temperature generated by an ultrasonic vibrator included in the ultrasonic surgical device; and
correcting frequency error information according to a change in temperature by adjusting frequency information on the basis of a difference between pre-stored temperature change frequency information and the identification frequency information, and
wherein any one probe is determined among the multiple types of probes in consideration of the corrected frequency information.

3. The method of claim 2, comprising:
before the receiving of the identification frequency, a tuning step of scanning a frequency to find a resonant frequency of a transducer included in the ultrasonic surgical device; and
a tracking step of maintaining the resonant frequency of the transducer, which is detected by scanning, as a predetermined resonant frequency,
wherein the receiving of the identification frequency comprises receiving identification frequency detected in the tuning step and the tracking step.

4. An apparatus for discriminating probes using a change in resonant frequency, the apparatus comprising:
a receiving unit configured to receive an identification frequency for discriminating the type of probe of an ultrasonic surgical device comprising a transducer and configured to generate ultrasonic waves by using an ultrasonic vibrator on the basis of a control signal;
an analyzing unit configured to analyze a feedback signal generated on the basis of the received identification frequency information;
a discriminating unit configured to discriminate any one probe among multiple types of probes on the basis of comparison between an analysis result and preset reference frequency information; and
a drive control unit configured to generate a control signal for controlling an operation of the ultrasonic surgical device on the basis of a predetermined value corresponding to the discriminated type of probe.

5. The apparatus of claim 4, wherein the discriminating unit comprises:
a temperature detecting unit configured to detect information on a temperature generated by the ultrasonic vibrator included in the ultrasonic surgical device; and
a correcting unit configured to correct frequency error information according to a change in temperature by adjusting frequency information on the basis of a difference between pre-stored temperature change frequency information and the identification frequency information, and
wherein any one probe is determined among the multiple types of probes in consideration of the corrected frequency information.
